(19) United States
(12) Patent Application Publication
(10) Pub. No.: US 2021/0110351 A1
(43) Pub. Date: Apr. 15, 2021

(54) SYSTEMS AND METHODS FOR A BENEFIT RECOMMENDATION ENGINE AND MEDICAL PLAN DECISION SUPPORT TOOL

(71) Applicant: FMR LLC, Boston, MA (US)

(72) Inventors: Xiaohua Wang, Lexington, MA (US); Victor Lo, Lexington, MA (US); Charles Breen, Windham, NH (US); Amol Jadhav, Bangalore (IN); Radha N, Bellary (IN); Avishek Pradhan, Bangalore (IN); Rommel Espinal, Amherst, NH (US)

(21) Appl. No.: 17/066,301

(22) Filed: Oct. 8, 2020

Related U.S. Application Data
(60) Provisional application No. 62/914,091, filed on Oct. 11, 2019.

(51) Int. Cl. G06Q 10/10; G16H 70/40; G16H 70/20; G16H 10/20; G16H 50/70; G16H 50/20; G16H 40/20; G06Q 30/00; G06Q 10/06; G16H 10/60; G06Q 30/02; G16H 15/00; G06Q 40/08; G06F 16/2457

(52) U.S. Cl. CPC G06Q 10/1057; G06F 16/24575; G16H 70/20; G16H 10/20; G16H 50/70; G16H 50/20; G16H 40/20; G06Q 30/0185; G06Q 10/06375; G16H 10/60; G06Q 30/0205; G16H 15/00; G06Q 10/06393; G06Q 40/08; G16H 70/40

(57) ABSTRACT

Systems and methods for determining an optimized medical plan using a machine learning-based model. The method includes receiving benchmarking data from a database including commercial claims data and past expense data. The method further includes receiving participant data from a user interface on a user device. The participant data includes demographic data, geographic data, and a past health service profile. The method also includes determining eligibility data and calculating base expense data based on the benchmarking data and the participant data. The method further includes calibrating the base expense data using the past expense data and a linear model. The method also includes calculating predicted expense data based on the calibrated base expense data. The method further includes determining medical plan configurations based on the predicted expense data and the eligibility data. The method also includes generating for display the medical plan configurations on the user device.

### CROSS REFERENCE TO RELATED APPLICATION

This application claims the benefit of and priority to U.S. Provisional Patent Application No. 62/914,091, filed Oct. 11, 2019, the entire contents of which are owned by the assignee of the instant application and incorporated herein by reference in their entirety.

### FIELD OF THE INVENTION

The present invention relates generally to systems and methods for a machine learning-based benefit recommendation engine, including systems and methods for determining an optimized medical plan using an artificial intelligence, machine learning-based model.

### BACKGROUND OF THE INVENTION

In general, it is challenging for average consumers or employees to foresee their next year health service needs. Some of the common factors employees consider during health plan selection include premium costs and costs at point of service, i.e., out-of-pocket costs. Often, employees are not confident when selecting a health plan because of a struggle to link clinical risk and financial risk shared with insurance carriers. As a result, financial-capable employees tend to over insure and a majority of employees tend to stay with their original medical plan. A lack of a decision support tool leads to unnecessary financial waste to both employees and employers. Accordingly, there is a need for a platform that helps participants select the right medical plan and health savings account contribution among various sources.

### SUMMARY OF THE INVENTION

Accordingly, an object of the invention is to provide users with systems and methods for a benefit recommendation engine and medical plan decision support tool. It is an object of the invention to provide users with systems and methods for predicting future out-of-pocket expenses. It is an object of the invention to provide users with systems and methods for providing benefit recommendations using an artificial intelligence, machine learning-based model. It is an object of the invention to provide users with systems and methods for determining an optimized medical plan using a machine learning-based model.

In some aspects, a method for determining an optimized medical plan using a machine learning-based model includes receiving, by a server computing device, benchmarking data from a database. The benchmarking data includes commercial claims data and past expense data. The method further includes receiving, by the server computing device, participant data from a user interface on a user device. The participant data includes demographic data, geographic data, and a past health service profile. The method also includes determining, by the server computing device, eligibility data based on the benchmarking data and the participant data.

Further, the method includes calculating, by the server computing device, base expense data based on the benchmarking data and the participant data using a spline regression model. The method also includes calibrating, by the server computing device, the base expense data using the past expense data and a linear model. The method further includes calculating, by the server computing device, predicted expense data based on the calibrated base expense data. Further, the method includes determining, by the server computing device, medical plan configurations based on the predicted expense data and the eligibility data. The method also includes generating, by the server computing device, for display the medical plan configurations on the user device.

In some embodiments, the benchmarking data includes medical and prescription drug data. In other embodiments, the demographic data includes at least one of an age of the participant or a covered family size. In some embodiments, the past health service profile includes at least one of medical history, pharmaceutical history, or history of chronic conditions.

In other embodiments, the server computing device is further configured to receive adjusted contribution data from the user interface on the user device. For example, in some embodiments, the server computing device is further configured to determine adjusted medical plan configurations based on the predicted expense data, the eligibility data, and the adjusted contribution data. In some embodiments, the server computing device is further configured to generate for display the adjusted medical plan configurations on the user device.

In some aspects, a system for determining an optimized medical plan using a machine learning-based model includes a server computing device communicatively coupled to a database and a user device over a network. The server computing device is configured to receive benchmarking data from the database. The benchmarking data includes commercial claims data and past expense data. The server computing device is also configured to receive participant data from a user interface on the user device. The participant data includes demographic data, geographic data, and a past health service profile. Further, the server computing device is configured to determine eligibility data based on the benchmarking data and the participant data.

Further, the server computing device is configured to calculate base expense data based on the benchmarking data and the participant data using a spline regression model. The server computing device is also configured to calibrate the base expense data using the past expense data and a linear model. The server computing device is also configured to calculate predicted expense data based on the calibrated base expense data. The server computing device is also configured to determine medical plan configurations based on the predicted expense data and the eligibility data. Further, the server computing device is configured to generate for display the medical plan configurations on the user device.

In some embodiments, the benchmarking data includes medical and prescription drug data. In other embodiments, the demographic data includes at least one of an age of the participant or a covered family size. In some embodiments, the past health service profile includes at least one of medical history, pharmaceutical history, or history of chronic conditions.

In other embodiments, the server computing device is further configured to receive adjusted contribution data from the user interface on the user device. For example, in some embodiments, the server computing device is further configured to determine adjusted medical plan configurations based on the predicted expense data, the eligibility data, and the adjusted contribution data. In some embodiments, the server computing device is further configured to generate for display the adjusted medical plan configurations on the user device.

In some aspects, a method for determining an optimized medical plan using a machine learning-based model includes receiving, by a server computing device, first benchmarking data and second benchmarking data from a database. The first benchmarking data is associated with medical data and the second benchmarking data is associated with pharmaceutical data. The method further includes receiving, by the server computing device, participant data from a user interface on a user device. The method also includes determining, by the server computing device, eligibility data based on the first benchmarking data, the second benchmarking data, and the participant data.

Further, the method includes calculating, by the server computing device, base expense data based on the first benchmarking data, the second benchmarking data, and the participant data using a spline regression model. The method also includes calibrating, by the server computing device, the base expense data using past expense data and a linear model. The method further includes calculating, by the server computing device, predicted expense data based on the calibrated base expense data. Further, the method includes determining, by the server computing device, medical plan configurations based on the predicted expense data and the eligibility data. The method also includes receiving, by the server computing device, adjusted contribution data from the user interface on the user device. Further, the method includes determining, by the server computing device, adjusted medical plan configurations based on the predicted expense data, the eligibility data, and the adjusted contribution data.

In some embodiments, the participant data includes demographic data, geographic data, and a past health service profile. For example, in some embodiments, the demographic data includes at least one of an age of the participant or a covered family size.

In some embodiments, the past health service profile includes at least one of medical history, pharmaceutical history, or history of chronic conditions. For example, in some embodiments, the server computing device is further configured to determine a major diagnostic category based on the past health service profile.

In other embodiments, the server computing device is further configured to generate for display the medical plan configurations and the adjusted medical plan configurations on the user device.

Other aspects and advantages of the invention can become apparent from the following drawings and description, all of which illustrate the principles of the invention, by way of example only.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1 is a block diagram of an exemplary data communications network according to embodiments of the technology described herein.

FIG. 2 is a block diagram of an exemplary server computing device and an exemplary user device according to embodiments of the technology described herein.

FIG. 3 is a diagram demonstrating an exemplary architecture for determining an optimized medical plan using a machine learning-based model, according to embodiments of the technology described herein.

FIG. 4 is a diagram demonstrating an exemplary process flow for determining an optimized medical plan using the exemplary architecture shown in FIG. 3, according to embodiments of the technology described herein.

FIG. 5 is a flow diagram of a computer-implemented method for determining an optimized medical plan using the exemplary architecture shown in FIG. 3, according to embodiments of the technology described herein.

FIG. 6 is a flow diagram of a computer-implemented method for determining an optimized medical plan using the exemplary architecture shown in FIG. 3, according to embodiments of the technology described herein.

### DETAILED DESCRIPTION OF THE INVENTION

The technology described herein is capable of providing users with systems and methods for a benefit recommendation engine and medical plan decision support tool. The technology described herein is capable of providing users with systems and methods for predicting future out-of-pocket expenses. The technology described herein is capable of providing users with systems and methods for providing benefit recommendations using an artificial intelligence, machine learning-based model. The technology described herein is capable of providing users with systems and methods for determining an optimized medical plan using a machine learning-based model.

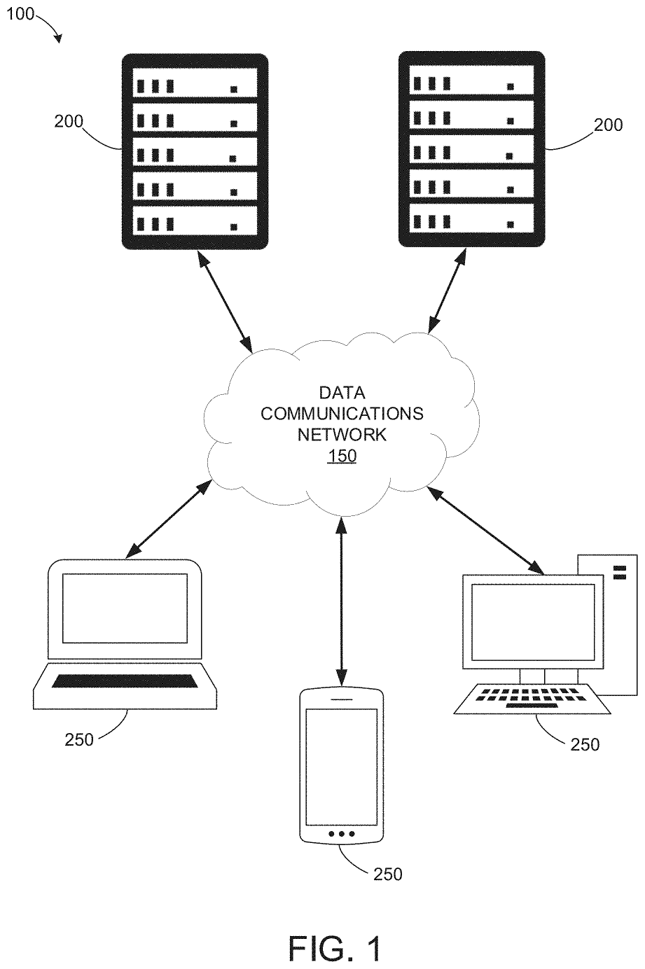

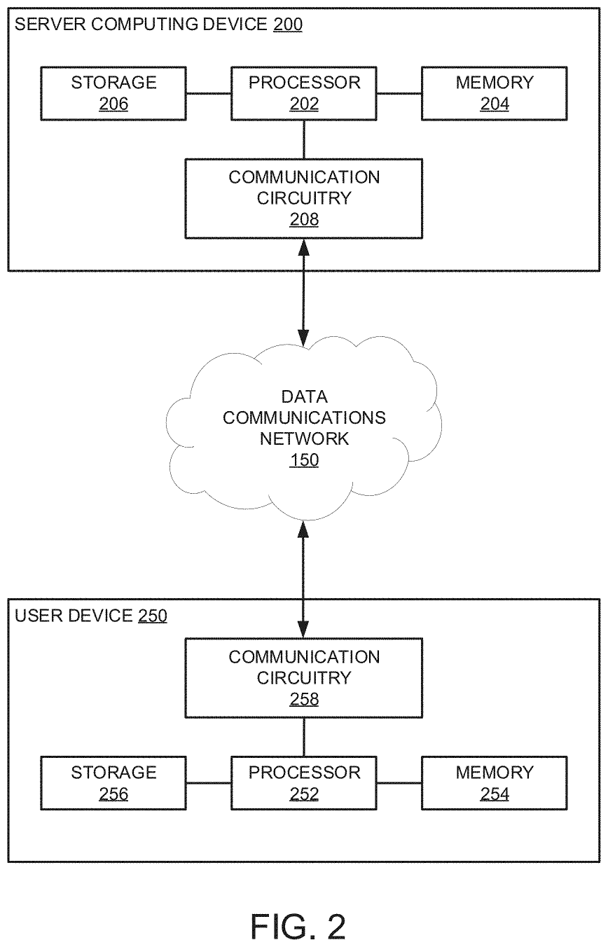

Referring to FIGS. 1 and 2, an exemplary communications system 100 includes data communications network 150, exemplary server computing devices 200, and exemplary user devices 250. In some embodiments, the system 100 includes one or more server computing devices 200 and one or more user devices 250. Each server computing device 200 can include a processor 202, memory 204, storage 206, and communication circuitry 208. Each user device 250 can include a processor 252, memory 254, storage 256, and communication circuitry 258. In some embodiments, communication circuitry 208 of the server computing devices 200 is communicatively coupled to the communication circuitry 258 of the user devices 250 via data communications network 150. Communication circuitry 208 and communication circuitry 258 can use Bluetooth, Wi-Fi, or any comparable data transfer connection. The user devices 250 can include smartphones, personal workstations, laptops, tablets, mobile devices, or any other comparable device.

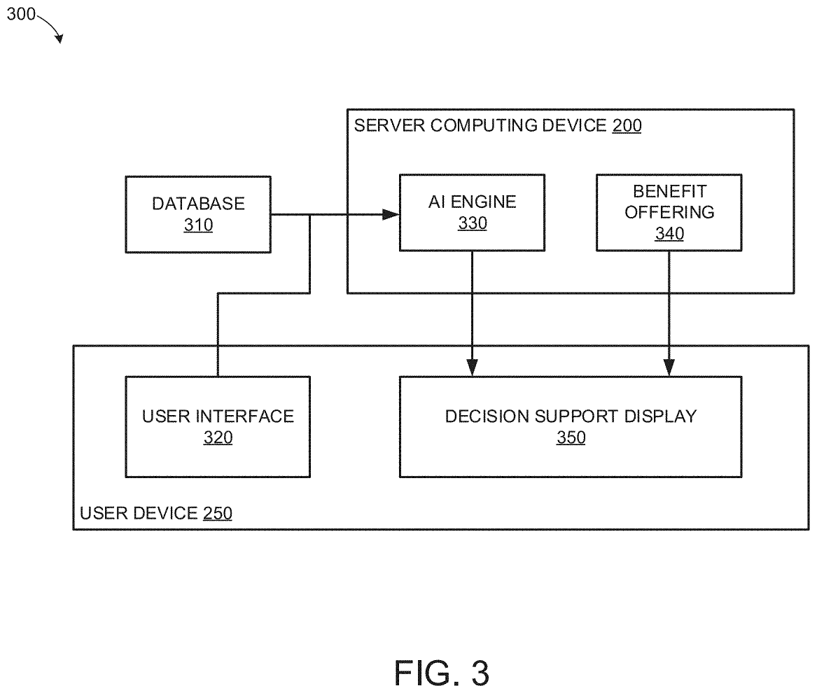

The medical plan decision support tool (“MDST”) is a platform that helps participants select the right medical plan and health savings account (“HSA”) contribution among various available choices. Referring to FIG. 3, the MDST architecture 300 processes input data from one or more databases 310 and responses filled by participants using a user interface 320 on a user device 250. The MDST platform 300 includes an AI engine 330 and a benefit offering module 340 which create an output displaying estimated future out-of-pocket expenses and eligible medical plan configurations on a decision support display 350. In some embodiments, the decision support display 350 can be on the same user device 250 or on a different user device 250.

The AI engine 330 utilizes advanced machine learning and deep learning techniques along with benchmarking data and information provided by the participant. For example, in some embodiments, the benchmarking data can include commercial claims from medical and pharmaceutical databases. The information provided by the participant can include demographic information, geographic information, and a past health service profile that can contain information such as medical history, pharmaceutical history, and any history of chronic conditions. The MDST architecture 300 uses the benchmarking data and participant information to predict and calculate future out-of-pocket expenses and provide recommended medical plan configurations.

In some embodiments, the MDST architecture 300 includes a claims data module that receives and collects user's claims data in order to predict and calculate future out-of-pocket expenses and provide recommended medical plan configurations instead of relying on participant information provided by the user. In other embodiments, the MDST architecture 300 includes a health comparison tool that enables users to have more confidence in choosing among the plans offered. For example, the health comparison tool enables users to view plan details and make comparisons for relevant attributes such as deductible, copay, prescription drug costs, etc. In some embodiments, the MDST architecture 300 includes a plan and learn resource module that provides additional descriptive information on health plan types and key features. The plan and learn resource module mitigates the impact of the lack of healthcare and health financial knowledge.

In some embodiments, the MDST architecture 300 includes a provider smart search module that identifies in-network providers by connecting to third party provider databases. In some embodiments, the MDST architecture 300 includes a life event module that identifies life events that trigger re-enrollment. For example, in some embodiments, new hire and other life events can trigger enrollment outside of the usual enrollment period. The life event module can expand the functionality of the MDST architecture 300 for eligible users, including adjusting total costs due to the life event.

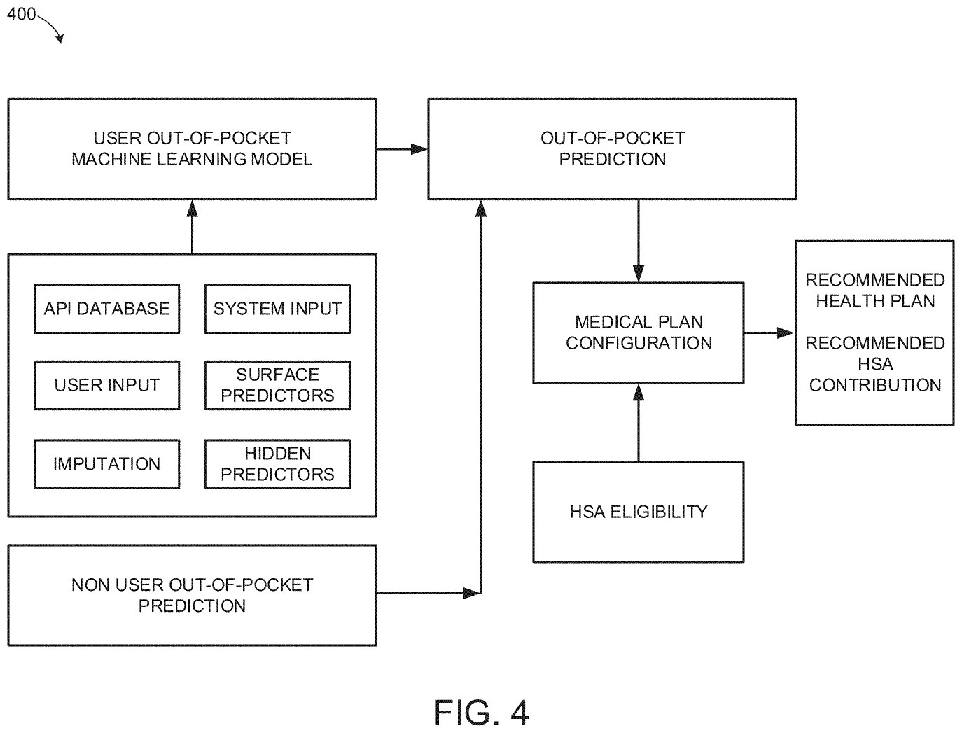

FIG. 4 illustrates the logic flow of an exemplary MDST machine learning model 400. The MDST model 400 can be implemented using supervised learning and/or machine learning algorithms. Supervised learning is the machine learning task of learning a function that maps an input to an output based on example input-output pairs. It infers a function from labeled training data consisting of a set of training examples. Each example is a pair consisting of an input object and a desired output value. A supervised learning algorithm or machine learning algorithm analyzes the training data and produces an inferred function, which can be used for mapping new examples.

For example, the exemplary MDST model 400 receives the benchmarking data and participant information from databases and user interfaces, respectively, and calculates a base out-of-pocket expense total based on the data. In some embodiments, the base out-of-pocket expense total is calculated using a spline regression model. For example, in some embodiments, the base out-of-pocket expense total is calculated by applying a Box-Cox transformation to a dependent variable, i.e., future out-of-pocket costs, and applying spline transformation to continuous features, while other categorical variables are dichotomized.

However, in some embodiments, the base out-of-pocket expense total is under-predicted because the model is under-predicted when a Box-Cox transformation is used. Therefore, the MDST model 400 then applies a calibration model to remove the bias from the base out-of-pocket expense total, using a technique similar to weather forecast calibration. In some embodiments, the calibration model is a linear model that uses past out-of-pocket expense data to calibrate the MDST model 400. For example, in some embodiments, the predicted output from the base model can be transformed into the original scale and new predictors can be derived using square and square root transformation. In some embodiments, the base and calibration models can be combined into a product model with joined features. By combining the base and calibration models into a product model, computing efficiency and integrity is enhanced.

As illustrated in FIG. 4, once the MDST model 400 has been calibrated, the model 400 can calculate a predicted out-of-pocket cost. The MDST model 400 uses the predicted out-of-pocket cost and HSA eligibility of the participant to assemble various medical plan configurations optimized for the participant. For example, the MDST model 400 can provide an optimal medical plan and HSA contribution that reduces the total cost for the participant, including premium cost and out-of-pocket expenses. The MDST model 400 allows a participant to customize their selection options by, for example, adjusting the HSA contribution. In some embodiments, the MDST model 400 adjusts the calculated predicted out-of-pocket cost using consumer price index data from the Bureau of Labor Statistics.

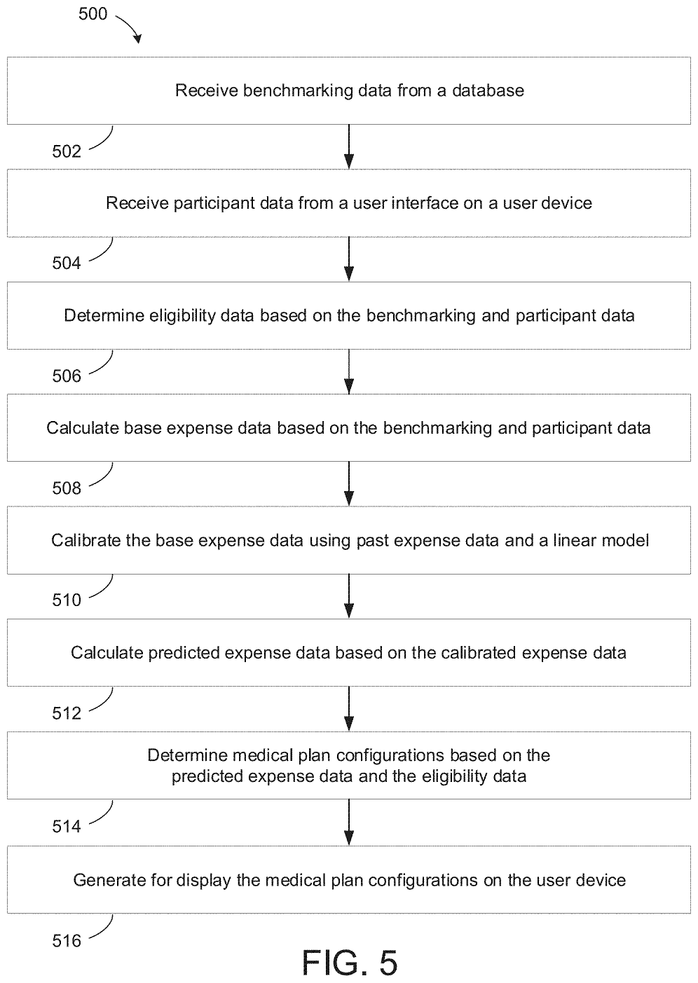

Referring to FIG. 5, a process 500 for determining an optimized medical plan using a machine learning-based model is illustrated. The process 500 begins by receiving benchmarking data from a database 310 in step 502. For example, in some embodiments, the benchmarking data includes commercial claims data and past expense data. In other embodiments, the benchmarking data includes medical and prescription drug data.

Process 500 continues by receiving participant data from a user interface 320 on a user device 250 in step 504. For example, in some embodiments, the participant data includes demographic data, geographic data, and a past health service profile. In some embodiments, the demographic data includes at least one of an age of the participant or a covered family size. In some embodiments, the past health service profile includes at least one of medical history, pharmaceutical history, or history of chronic conditions.

Process 500 continues by determining eligibility data based on the benchmarking data and the participant data in step 506. For example, in some embodiments, benefit offering module 340 can be configured to determine the eligibility data based on the benchmarking data and the participant data. Process 500 continues by calculating base expense data based on the benchmarking data and the participant data using a spline regression model in step 508. For example, in some embodiments, AI engine 330 can be configured to calculate the base expense data based on the benchmarking data and the participant data.

Process 500 continues by calibrating the base expense data using the past expense data and a linear model in step 510. For example, in some embodiments, AI engine 330 can be configured to calibrate the base expense data using the past expense data and a linear model. Process 500 continues by calculating predicted expense data based on the calibrated base expense data in step 512. For example, in some embodiments, AI engine 330 can be configured to calculate the predicted expense data based on the calibrated expense data.

Process 500 continues by determining medical plan configurations based on the predicted expense data and the eligibility data in step 514. For example, in some embodiments, AI engine 330 can be configured to determine the medical plan configurations based on the predicted expense data and the eligibility data. Process 500 finishes by generating for display the medical plan configurations on the user device 250 in step 516. For example, in some embodiments, the medical plan configurations can be generated for display on the decision support display 350 of user device 250.

In some embodiments, process 500 continues by receiving adjusted contribution data from the user interface 320 on the user device 250. For example, in some embodiments, process 500 continues by determining adjusted medical plan configurations based on the predicted expense data, the eligibility data, and the adjusted contribution data. In some embodiments, process 500 continues by generating for display the adjusted medical plan configurations on the decision support display 350 of user device 250.

In some aspects, process 500 can be implemented on a system for determining an optimized medical plan using a machine learning-based model. For example, the system can include a server computing device 200 communicatively coupled to a database 310 and a user device 250 over a network 150. The server computing device 200 is configured to receive benchmarking data from the database 310. The benchmarking data includes commercial claims data and past expense data. In some embodiments, the benchmarking data includes medical and prescription drug data. The server computing device 200 is also configured to receive participant data from a user interface 320 on the user device 250. The participant data includes demographic data, geographic data, and a past health service profile. In some embodiments, the demographic data includes at least one of an age of the participant or a covered family size. In some embodiments, the past health service profile includes at least one of medical history, pharmaceutical history, or history of chronic conditions.

Further, the server computing device 200 is configured to determine eligibility data based on the benchmarking data and the participant data. The server computing device 200 is also configured to calculate base expense data based on the benchmarking data and the participant data using a spline regression model. The server computing device 200 is also configured to calibrate the base expense data using the past expense data and a linear model. Further, the server computing device 200 is configured to calculate predicted expense data based on the calibrated base expense data. The server computing device 200 is also configured to determine medical plan configurations based on the predicted expense data and the eligibility data. Further, the server computing device 200 is configured to generate for display the medical plan configurations on the user device 250.

In some embodiments, the server computing device 200 is further configured to receive adjusted contribution data from the user interface 320 on the user device 250. For example, in some embodiments, the server computing device 200 is further configured to determine adjusted medical plan configurations based on the predicted expense data, the eligibility data, and the adjusted contribution data. In some embodiments, the server computing device 200 is further configured to generate for display the adjusted medical plan configurations on the user device 250.

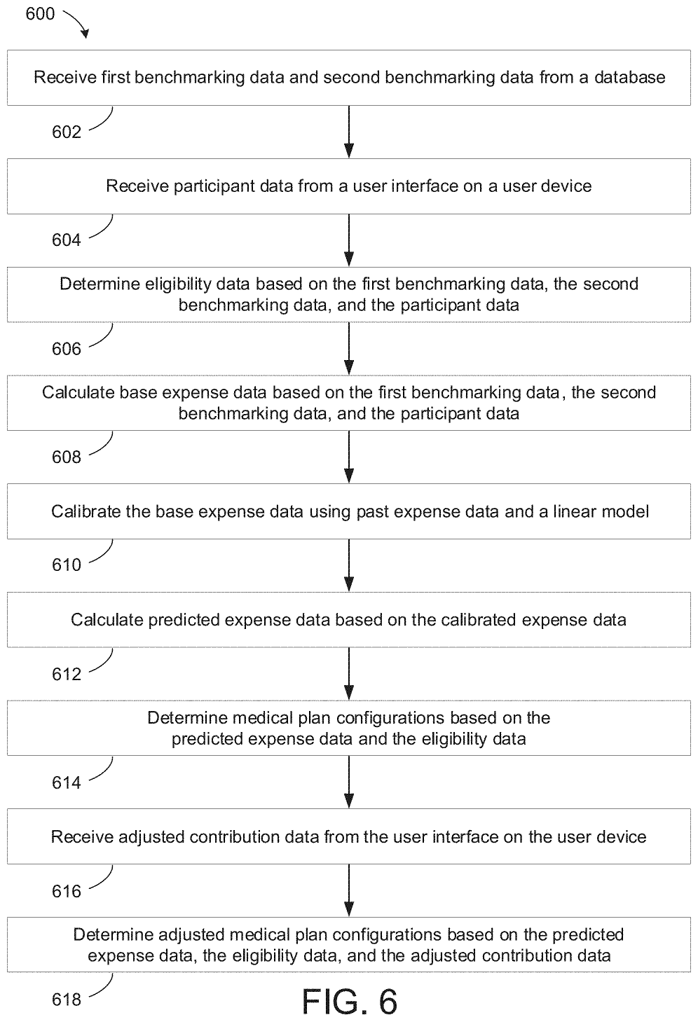

Referring to FIG. 6, a process 600 for determining an optimized medical plan using a machine learning-based model is illustrated. The process 600 begins by receiving first benchmarking data and second benchmarking data from a database 310 in step 602. For example, in some embodiments, the first benchmarking data is associated with medical data and the second benchmarking data is associated with pharmaceutical data. Process 600 continues by receiving participant data from a user interface 320 on a user device 250 in step 604. For example, in some embodiments, the participant data includes demographic data, geographic data, and a past health service profile. In some embodiments, the demographic data includes at least one of an age of the participant or a covered family size. In some embodiments, the past health service profile includes at least one of medical history, pharmaceutical history, or history of chronic conditions. In some embodiments, a major diagnostic category can be determined based on the past health service profile.

Process 600 continues by determining eligibility data based on the first benchmarking data, the second benchmarking data, and the participant data in step 606. For example, in some embodiments, benefit offering module 340 can be configured to determine the eligibility data based on the first benchmarking data, the second benchmarking data, and the participant data. Process 600 continues by calculating base expense data based on the first benchmarking data, the second benchmarking data, and the participant data using a spline regression model in step 608. For example, in some embodiments, AI engine 330 can be configured to calculate the base expense data based on the first benchmarking data, the second benchmarking data, and the participant data.

Process 600 continues by calibrating the base expense data using past expense data and a linear model in step 610. For example, in some embodiments, AI engine 330 can be configured to calibrate the base expense data using past expense data and a linear model. Process 600 continues by calculating predicted expense data based on the calibrated base expense data in step 612. For example, in some embodiments, AI engine 330 can be configured to calculate the predicted expense data based on the calibrated expense data. Process 600 continues by determining medical plan configurations based on the predicted expense data and the eligibility data in step 614. For example, in some embodiments, AI engine 330 can be configured to determine the medical plan configurations based on the predicted expense data and the eligibility data.

Process 600 continues by receiving adjusted contribution data from the user interface 320 on the user device 250 in step 616. Process 600 finishes by determining adjusted medical plan configurations based on the predicted expense data, the eligibility data, and the adjusted contribution data in step 618. In some embodiments, process 600 continues by generating for display the medical plan configurations and the adjusted medical plan configurations on the user device 250. For example, in some embodiments, the medical plan configurations and the adjusted medical plan configurations can be generated for display on the decision support display 350 of user device 250.

In some aspects, process 600 can be implemented on a system for determining an optimized medical plan using a machine learning-based model. For example, the system can include a server computing device 200 communicatively coupled to a database 310 and a user device 250 over a network 150. The server computing device 200 is configured to receive first benchmarking data and second benchmarking data from the database 310. The first benchmarking data is associated with medical data and the second benchmarking data is associated with pharmaceutical data. The server computing device 200 is also configured to receive participant data from a user interface 320 on the user device 250. In some embodiments, the participant data includes demographic data, geographic data, and a past health service profile. In some embodiments, the demographic data includes at least one of an age of the participant or a covered family size. In some embodiments, the past health service profile includes at least one of medical history, pharmaceutical history, or history of chronic conditions. In other embodiments, the server computing device 200 is also further configured to determine a major diagnostic category based on the past health service profile.

Further, the server computing device 200 is configured to determine eligibility data based on the first benchmarking data, the second benchmarking data, and the participant data. The server computing device 200 is also configured to calculate base expense data based on the first benchmarking data, the second benchmarking data, and the participant data using a spline regression model. The server computing device 200 is also configured to calibrate the base expense data using past expense data and a linear model. Further, the server computing device 200 is configured to calculate predicted expense data based on the calibrated base expense data. The server computing device 200 is also configured to determine medical plan configurations based on the predicted expense data and the eligibility data. Further, the server computing device 200 is configured to receive adjusted contribution data from the user interface 320 on the user device 250. The server computing device 200 is also configured to determine adjusted medical plan configurations based on the predicted expense data, the eligibility data, and the adjusted contribution data. In some embodiments, the server computing device 200 is further configured to generate for display the medical plan configurations and the adjusted medical plan configurations on the user device 250.

The above-described techniques can be implemented in digital and/or analog electronic circuitry, or in computer hardware, firmware, software, or in combinations of them. The implementation can be as a computer program product, i.e., a computer program tangibly embodied in a machine-readable storage device, for execution by, or to control the operation of, a data processing apparatus, e.g., a programmable processor, a computer, and/or multiple computers. A computer program can be written in any form of computer or programming language, including source code, compiled code, interpreted code and/or machine code, and the computer program can be deployed in any form, including as a stand-alone program or as a subroutine, element, or other unit suitable for use in a computing environment. A computer program can be deployed to be executed on one computer or on multiple computers at one or more sites. The computer program can be deployed in a cloud computing environment (e.g., Amazon® AWS, Microsoft® Azure, IBM®).

Method steps can be performed by one or more processors executing a computer program to perform functions of the invention by operating on input data and/or generating output data. Method steps can also be performed by, and an apparatus can be implemented as, special purpose logic circuitry, e.g., a FPGA (field programmable gate array), a FPAA (field-programmable analog array), a CPLD (complex programmable logic device), a PSoC (Programmable System-on-Chip), ASIP (application-specific instruction-set processor), or an ASIC (application-specific integrated circuit), or the like. Subroutines can refer to portions of the stored computer program and/or the processor, and/or the special circuitry that implement one or more functions.

Processors suitable for the execution of a computer program include, by way of example, special purpose microprocessors specifically programmed with instructions executable to perform the methods described herein, and any one or more processors of any kind of digital or analog computer. Generally, a processor receives instructions and data from a read-only memory or a random access memory or both. The essential elements of a computer are a processor for executing instructions and one or more memory devices for storing instructions and/or data. Memory devices, such as a cache, can be used to temporarily store data. Memory devices can also be used for long-term data storage. Generally, a computer also includes, or is operatively coupled to receive data from or transfer data to, or both, one or more mass storage devices for storing data, e.g., magnetic, magneto-optical disks, or optical disks. A computer can also be operatively coupled to a communications network in order to receive instructions and/or data from the network and/or to transfer instructions and/or data to the network. Computer-readable storage mediums suitable for embodying computer program instructions and data include all forms of volatile and non-volatile memory, including by way of example semiconductor memory devices, e.g., DRAM, SRAM, EPROM, EEPROM, and flash memory devices; magnetic disks, e.g., internal hard disks or removable disks; magneto-optical disks; and optical disks, e.g., CD, DVD, HD-DVD, and Blu-ray disks. The processor and the memory can be supplemented by and/or incorporated in special purpose logic circuitry.

To provide for interaction with a user, the above described techniques can be implemented on a computing device in communication with a display device, e.g., a CRT (cathode ray tube), plasma, or LCD (liquid crystal display) monitor, a mobile device display or screen, a holographic device and/or projector, for displaying information to the user and a keyboard and a pointing device, e.g., a mouse, a trackball, a touchpad, or a motion sensor, by which the user can provide input to the computer (e.g., interact with a user interface element). Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, and/or tactile input.

The above-described techniques can be implemented in a distributed computing system that includes a back-end component. The back-end component can, for example, be a data server, a middleware component, and/or an application server. The above described techniques can be implemented in a distributed computing system that includes a front-end component. The front-end component can, for example, be a client computer having a graphical user interface, a Web browser through which a user can interact with an example implementation, and/or other graphical user interfaces for a transmitting device. The above described techniques can be implemented in a distributed computing system that includes any combination of such back-end, middleware, or front-end components.

The components of the computing system can be interconnected by transmission medium, which can include any form or medium of digital or analog data communication (e.g., a communication network). Transmission medium can include one or more packet-based networks and/or one or more circuit-based networks in any configuration. Packet-based networks can include, for example, the Internet, a carrier internet protocol (IP) network (e.g., local area network (LAN), wide area network (WAN), campus area network (CAN), metropolitan area network (MAN), home area network (HAN)), a private IP network, an IP private branch exchange (IPBX), a wireless network (e.g., radio access network (RAN), Bluetooth, near field communications (NFC) network, Wi-Fi, WiMAX, general packet radio service (GPRS) network, HiperLAN), and/or other packet-based networks. Circuit-based networks can include, for example, the public switched telephone network (PSTN), a legacy private branch exchange (PBX), a wireless network (e.g., RAN, code-division multiple access (CDMA) network, time division multiple access (TDMA) network, global system for mobile communications (GSM) network), and/or other circuit-based networks.

Information transfer over transmission medium can be based on one or more communication protocols. Communication protocols can include, for example, Ethernet protocol, Internet Protocol (IP), Voice over IP (VOIP), a Peer-to-Peer (P2P) protocol, Hypertext Transfer Protocol (HTTP), Session Initiation Protocol (SIP), H.323, Media Gateway Control Protocol (MGCP), Signaling System #7 (SS7), a Global System for Mobile Communications (GSM) protocol, a Push-to-Talk (PTT) protocol, a PTT over Cellular (POC) protocol, Universal Mobile Telecommunications System (UMTS), 3GPP Long Term Evolution (LTE) and/or other communication protocols.

Devices of the computing system can include, for example, a computer, a computer with a browser device, a telephone, an IP phone, a mobile device (e.g., cellular phone, personal digital assistant (PDA) device, smartphone, tablet, laptop computer, electronic mail device), and/or other communication devices. The browser device includes, for example, a computer (e.g., desktop computer and/or laptop computer) with a World Wide Web browser (e.g., Chrome™ from Google, Inc., Microsoft® Internet Explorer® available from Microsoft Corporation, and/or Mozilla® Firefox available from Mozilla Corporation). Mobile computing device include, for example, a Blackberry® from Research in Motion, an iPhone® from Apple Corporation, and/or an Android™-based device. IP phones include, for example, a Cisco® Unified IP Phone 7985G and/or a Cisco® Unified Wireless Phone 7920 available from Cisco Systems, Inc.

The above-described techniques can be implemented using supervised learning and/or machine learning algorithms. Supervised learning is the machine learning task of learning a function that maps an input to an output based on example input-output pairs. It infers a function from labeled training data consisting of a set of training examples. Each example is a pair consisting of an input object and a desired output value. A supervised learning algorithm or machine learning algorithm analyzes the training data and produces an inferred function, which can be used for mapping new examples.

Comprise, include, and/or plural forms of each are open ended and include the listed parts and can include additional parts that are not listed. And/or is open ended and includes one or more of the listed parts and combinations of the listed parts.

One skilled in the art will realize the subject matter may be embodied in other specific forms without departing from the spirit or essential characteristics thereof. The foregoing embodiments are therefore to be considered in all respects illustrative rather than limiting of the subject matter described herein.

What is claimed:

1. A computerized method for determining an optimized medical plan using a machine learning-based model, the method comprising:
receiving, by a server computing device, first benchmarking data and second benchmarking data from a database, wherein the first benchmarking data is associated with medical data and the second benchmarking data is associated with pharmaceutical data;
receiving, by the server computing device, participant data from a user interface on a user device;
determining, by the server computing device, eligibility data based on the first benchmarking data, the second benchmarking data, and the participant data;
calculating, by the server computing device, base expense data based on the first benchmarking data, second benchmarking data, and the participant data using a spline regression model;
calibrating, by the server computing device, the base expense data using past expense data and a linear model;
calculating, by the server computing device, predicted expense data based on the calibrated base expense data;
determining, by the server computing device, a plurality of medical plan configurations based on the predicted expense data and the eligibility data;
receiving, by the server computing device, adjusted contribution data from the user interface on the user device; and
determining, by the server computing device, an adjusted plurality of medical plan configurations based on the predicted expense data, the eligibility data, and the adjusted contribution data.

2. The computerized method of claim 1, wherein the participant data further comprises demographic data, geographic data, and a past health service profile.

3. The computerized method of claim 2, wherein the demographic data comprises at least one of an age of the participant or a covered family size.

4. The computerized method of claim 2, wherein the past health service profile comprises at least one of medical history, pharmaceutical history, or history of chronic conditions.

5. The computerized method of claim 4, wherein the server computing device is further configured to determine a major diagnostic category based on the past health service profile.

6. The computerized method of claim 1, wherein the server computing device is further configured to generate for display the medical plan configurations and the adjusted plurality of medical plan configurations on the user device.

7. A computerized method for determining an optimized medical plan using a machine learning-based model, the method comprising:
receiving, by a server computing device, benchmarking data from a database, wherein the benchmarking data comprises commercial claims data and past expense data;
receiving, by the server computing device, participant data from a user interface on a user device, wherein the participant data comprises demographic data, geographic data, and a past health service profile;
determining, by the server computing device, eligibility data based on the benchmarking data and the participant data;
calculating, by the server computing device, base expense data based on the benchmarking data and the participant data using a spline regression model;
calibrating, by the server computing device, the base expense data using the past expense data and a linear model;
calculating, by the server computing device, predicted expense data based on the calibrated base expense data;
determining, by the server computing device, a plurality of medical plan configurations based on the predicted expense data and the eligibility data; and
generating, by the server computing device, for display the plurality of medical plan configurations on the user device.

8. The computerized method of claim 7, wherein the benchmarking data comprises medical and prescription drug data.

9. The computerized method of claim 7, wherein the demographic data comprises at least one of an age of the participant or a covered family size.

10. The computerized method of claim 7, wherein the past health service profile comprises at least one of medical history, pharmaceutical history, or history of chronic conditions.

11. The computerized method of claim 7, wherein the server computing device is further configured to receive adjusted contribution data from the user interface on the user device.

12. The computerized method of claim 11, wherein the server computing device is further configured to determine an adjusted plurality of medical plan configurations based on the predicted expense data, the eligibility data, and the adjusted contribution data.

13. The computerized method of claim 12, wherein the server computing device is further configured to generate for display the adjusted plurality of medical plan configurations on the user device.

14. A system for determining an optimized medical plan using a machine learning-based model, the system comprising:
a server computing device communicatively coupled to a database and a user device over a network, the server computing device configured to:
receive benchmarking data from the database, wherein the benchmarking data comprises commercial claims data and past expense data;
receive participant data from a user interface on the user device, wherein the participant data comprises demographic data, geographic data, and a past health service profile;
determine eligibility data based on the benchmarking data and the participant data;
calculate base expense data based on the benchmarking data and the participant data using a spline regression model;
calibrate the base expense data using the past expense data and a linear model;
calculate predicted expense data based on the calibrated base expense data;
determine a plurality of medical plan configurations based on the predicted expense data and the eligibility data; and
generate for display the plurality of medical plan configurations on the user device.

15. The system of claim 14, wherein the benchmarking data comprises medical and prescription drug data.

16. The system of claim 14, wherein the demographic data comprises at least one of an age of the participant or a covered family size.

17. The system of claim 14, wherein the past health service profile comprises at least one of medical history, pharmaceutical history, or history of chronic conditions.

18. The system of claim 14, wherein the server computing device is further configured to receive adjusted contribution data from the user interface on the user device.

19. The system of claim 18, wherein the server computing device is further configured to determine an adjusted plurality of medical plan configurations based on the predicted expense data, the eligibility data, and the adjusted contribution data.

20. The system of claim 19, wherein the server computing device is further configured to generate for display the adjusted plurality of medical plan configurations on the user device.
